(19) United States
(12) Patent Application Publication
(10) Pub. No.: US 2026/0284312 A1
(43) Pub. Date: Sep. 24, 2026

(54) Medicament delivery device and medicament delivery assembly

(71) Applicant: SHL Medical AG, Zug (CH)

(72) Inventors: Justin Stewart, Deerfield Beach, FL (US); Andrew Moore, Zug (CH)

(21) Appl. No.: 19/477,939

(22) PCT Filed: Apr. 12, 2024
(86) PCT No.: PCT/EP2024/059957

§ 371 Date: Oct. 23, 2025

Related U.S. Application Data
(60) Provisional application No. 63/462,287, filed on Apr. 27, 2023.

(30) Foreign Application Priority Data
Jul. 27, 2023 (EP) 23188165.7

(51) Int. Cl. A61M 5/20; A61M 5/315

(52) U.S. Cl. CPC A61M 5/2033; A61M 5/31501; A61M 5/31578; A61M 2005/2013; A61M 2202/0007; A61M 2205/581

(57) ABSTRACT

The present disclosure provides a medicament delivery device for accommodating a medicament container. The device comprises a distal cap, a plunger rod, a signal member having a distal base and a plurality of arms extending proximally therefrom along the plunger rod, a drive spring extending at least partially into an interior of the plunger rod, the drive spring having a proximal end abutting against a proximal end bottom of the plunger rod and a distal end abutting against the distal base of the signal member. The signal member, the plunger rod and the biasing member are at least partially accommodated in a cavity of the distal cap, said cavity being open towards proximally. In an initial state the drive spring is compressed and there is a gap between the distal base of the signal member and a distal end wall of the cavity. A release of the drive spring moves the plunger rod relative to the distal cap into a proximal direction, causing the plunger rod to act on the medicament container for expelling a medicament therefrom, and moves the signal member relative to the distal cap into a distal direction,

### TECHNICAL FIELD

The invention is in the field of medicament delivery devices. More in particular, it relates to an automatic medicament delivery device providing audible, tactile and/or haptic feedback to a patient who uses it. It further relates to an assembly comprising such a device, pre-assembled with a medicament container.

### BACKGROUND

Medicament delivery devices for automatic delivery of a medicament by self-administration are well-known. Especially, they may be equipped to accommodate a medicament container, for example a medicament container with a septum to be perforated immediately prior to use, or a syringe. Often, the medicament delivery device and the medicament container are pre-assembled to constitute a medicament delivery assembly for self-administration.

Such medicament delivery devices and medicament delivery assemblies should generally be safe to use and easy to handle, and they should ensure delivery of the designated dose of the medicament. In view of this, it may be important that the patient receives a feedback signal during and/or after delivery of the medicament.

WO 2011/123024 discloses a medicament delivery device for a syringe as a medicament container, wherein the medicament delivery device provides an audible, tactile or visible confirmation to a patient when an injection has been made. To this end, the medicament delivery device comprises a signal generating member being an elongated U-bracket. The U-bracket is allowed to be displaced towards distally as soon as the plunger rod that displaces the plunger of the syringe is in the end position. The drive spring, which displaces the plunger rod during delivery, then acts to move the U-bracket towards distally until it taps against the inner surface of the housing, resulting in the desired audible signal.

In practice, the timing of the audible signal may be an issue. Due to required tolerances, it is virtually impossible to ensure that the medicament delivery process is completely finished at the moment the U-bracket is allowed to be displaced for generating the audible signal. Therefore, to safely ensure that there will be a confirmation in any case, the mechanism has to be activated a bit before the medicament delivery process is completely over. Thus, the patient hears the confirmation of the finished medicament delivery often a bit too early.

### SUMMARY

Accordingly, it is an object of the present invention to provide a medicament delivery device that overcomes at least some of the disadvantages of the prior art. Especially, it is an object of the invention to provide a medicament delivery device that improves the timing of the audible signal.

These and other objects are achieved by the invention as defined in the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

When a component is said to move proximally, distally, axially in a proximal direction, axially in a distal direction, in a circumferential direction, or equivalent terms, the movement is relative to the housing of the injection device, unless mentioned otherwise.

According to an aspect of the present invention, a medicament delivery device for accommodating a medicament container is provided.

The medicament delivery device comprises
a distal cap,
a plunger rod,
a signal member having a distal base and a plurality of arms extending proximally therefrom along the plunger rod,
a drive spring extending at least partially into an interior of the plunger rod, the drive spring having a proximal end abutting against a proximal end bottom of the plunger rod and a distal end abutting against the distal base of the signal member,

The signal member, the plunger rod and the biasing member are at least partially accommodated in a cavity of the distal cap, said cavity being open towards proximally.

In an initial state the drive spring is compressed and there is a gap between the distal base of the signal member and a distal end wall of the cavity. A release of the drive spring:
moves the plunger rod relative to the distal cap into a proximal direction, causing the plunger rod to act on the medicament container for expelling a medicament therefrom, and
moves the signal member relative to the distal cap into a distal direction.

The medicament delivery device further comprises a delay mechanism comprising:
a rod extending proximally in the cavity from the distal end wall, through an orifice of the distal base of the signal member,
a delay member accommodated in the gap at least in the initial state, around a section of the rod.

If the medicament container has a plunger or stopper, the plunger rod may for example act, by being moved into the proximal direction, on the plunger or stopper to expel the medicament via a needle, which needle may belong to the medicament delivery device, or, for example if the medicament container is a syringe, to the medicament container.

The delay mechanism delays the distal movement of the signal member. The delay mechanism prevents the signal member from being subject to the acceleration that the drive spring would cause to the signal member if it could be moved to the end point of its distal movement without any obstacles. A delay mechanism thus implies that the distal movement is obstructed/slowed down in some way.

In a first embodiment, the rod comprises a first section having an outer thread, and the delay member comprises an inner thread interfacing the outer thread, wherein during the release of the drive spring, the distal movement of the signal member causes the delay member to rotate around the first section and move distally along the rod up to the distal end wall, causing an audible signal.

The screw interface between the delay member and the rod causes the delay member to rotate when the delay member is cause to travel, when, due to the distal movement of the signal member, the signal member urges it towards distally. This delays the distal movement. It is then the delay member that taps against the distal end wall to cause the audible signal.

The delay mechanism may be configured to act during a first part of the distal movement and to not act during second part of the distal movement. In other words, the distal movement may be delayed by the delay mechanism while the signal member travels along a first section of its displacement path and may be not delayed along a second section thereof. Thus, the delay mechanism may comprise an obstruction means acting along the first section. Along the second section, the signal member is allowed to be accelerated, especially by the drive spring that also causes the proximal movement of the plunger rod, until the distal end wall of the distal cap stops it, whereby an audible signal is generated.

Hence, the rod my comprise a second section that is non-threaded, between the distal end wall and the first section.

The delay member may be a ring.

In a second embodiment, the delay member is an enlarged area of the section of the rod, and during the release of the drive spring, the distal movement of the signal member causes the orifice to slide over the delay member in a manner that friction is caused, the distal base moving distally along the rod up to the distal end wall, causing an audible signal.

An enlarged area in this sense may be a widening feature, thus a feature where the outer circumference of the rod, perpendicular to the proximodistal axis, is enhanced compared to other. In the second embodiment, there is no substantial rotation of the delay member, nor an axial displacement of the delay member along the rod. The orifice may be slightly undersized so that there will be significant friction between the delay member and the orifice. Because of the orifice being slightly undersized, a (slight) elastic deformation is required for the displacement movement to take place. This will cause the delay. The orifice sliding over the delay member may cause an elastic deformation of the base unit and/or the delay member.

The delay member may be bulb-like, and the orifice may be circular.

To increase elasticity, the signal member may optionally comprise a plurality of cuts extending radially outwardly from the orifice.

Alternatively, the signal member may comprise an oversized orifice (compared to the radial dimensions of the delay member), with at least one inwardly facing tongue that needs to slide over the delay member during the distal movement, requiring some elastic deformation of the tongue(s).

The following refers to both the first and the second embodiments.

In the initial state, the distal base of the signal member may be in contact with the delay member.

The rod may extend partially inside the drive spring.

The signal member may have the shape of a U-bracket, and at a proximal end of each arm, the signal member may have a radially outwardly extending, flange like, support portion.

In particular, the signal member and its working principle may be of the kind described in WO 2011/123024.

The signal member in the initial state—prior to the distal movement—may extend along at least a substantial portion of the plunger rod's axial extension. For example, if the signal member has the plurality of arms extending from the distal base, the plunger rod may be at least partially accommodated between the arms in the initial state. Thus, the arms extend along different sides of the plunger rod—for example along opposite sides if there are two arms.

The signal member may comprise outwardly protruding support portions—for example one for every arm—that secure the signal member from being pushed, by the drive spring, further into the cavity. For example, the support portions may be arranged immediately proximally of a mouth of the cavity, abutting against a proximal end of the tubular portion.

The audible signal may be initiated by the plunger rod moving out of the space between the arms of the signal member, i.e. the distal end portion of the plunger rod moving past the proximal end of the signal member. The position it reaches when it has moved out of the space between the arms of the signal member in these embodiments corresponds to the above-mentioned dispense position of the plunger rod. Especially, the arms may be biased towards radially-inwardly, pressing against the surface of the plunger rod. As soon as the plunger rod has towards the end of the process of expelling the medicament, moved out of the space between the arms of the signal member, the arms are allowed to flex radially-inwardly. As a result, the outwardly protruding support portions get out of engagement with the tubular portion's mouth, so that the signal member can be pushed, by the drive spring that urges the plunger rod towards proximally, against a resistance by the medicament container, towards distally, further into the cavity, to close the gap. The delay mechanism then acts to delay this displacement movement that closes the gap.

The distal cap may have a head portion comprising the distal end wall, and a tubular portion comprising the cavity.

The distal cap may form part of a housing of the medicament delivery device, for example together with a housing sleeve (thus, a tubular element of the housing). In addition to comprising a housing sleeve and/or a distal cap, the housing may comprise a container housing accommodated within the sleeve and providing a seat for the medicament container.

The medicament delivery device may comprise a drive locking member that, in the initial state, impedes a movement of the plunger rod towards proximally, and when activated, ceases to impede said movement, causing the drive springe to expand.

Initial activation may, as is known from the prior art, be automatic. For example, the medicament delivery device may comprise a needle guard protecting a needle (of the medicament delivery device or of the medicament container, the needle being for injecting the expelled medicament under the patient's skin) after a proximal cap or similar has been removed, immediately prior to use. The automatic release may be caused by the needle guard being displaced, against a biasing force, towards distally when the patient presses the medicament delivery assembly against the dose delivery site. To this end, the medicament delivery device may comprise a drive locking member that prevents the plunger rod from moving towards proximally prior to release. Release may for example be due to a rotation of the drive locking member, such rotation caused by the displacement of the needle guard towards distally.

In addition to concerning a medicament delivery device, the present invention also concerns a medicament delivery assembly comprising the medicament delivery device of the kind described in the present text, pre-assembled with a medicament container, for example a medicament container with a septum to be pierced by a needle of the medicament delivery device prior to use, or a syringe with a pre-assembled needle.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

The drawings show:

FIG. 1 a view of an embodiment of a medicament delivery assembly comprising the medicament delivery device and a medicament container;

FIG. 2 a housing sleeve of the medicament delivery device;

FIG. 3 a needle guard of the medicament delivery device;

FIG. 4 the medicament delivery assembly of FIG. 1, with a proximal cap and a needle cap assembly removed, and without the housing sleeve and the needle guard, the medicament container and a container housing being shown transparent;

FIG. 5 a sub-assembly of the medicament delivery assembly, the sub-assembly comprising a plunger rod, a signal member, a drive spring being a biasing member, and a delay member;

FIG. 6 the sub-assembly of FIG. 5 without the plunger rod;

FIG. 7 the delay member;

FIG. 8 a distal cap of the medicament delivery assembly;

FIGS. 9 and 10 partially, the sub-assembly of FIG. 5 assembled with the distal cap, in partial longitudinal section, during two different stages at the end of the medicament delivery process;

FIG. 11 a sub-assembly of an alternative embodiment of the medicament delivery assembly, the sub-assembly comprising a plunger rod, a signal member and a drive spring being a biasing member; and

FIG. 12 the sub-assembly of FIG. 11 assembled with a corresponding distal cap, in section.

### DETAILED DESCRIPTION

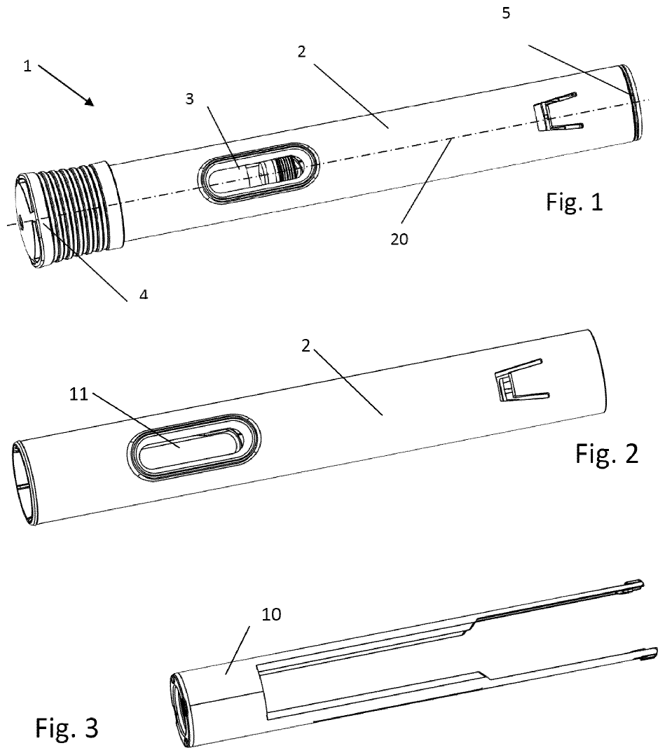

The medicament delivery assembly 1 shown in FIG. 1 comprises a medicament delivery device and a medicament container 3. The medicament delivery device comprises a base unit with a housing, the housing comprising a housing sleeve 2 and a distal cap 5. A proximal cap 4 protects, possibly together with a needle cap assembly that comprises at least one needle cap, the needle of the medicament delivery assembly during transportation and (short-time) storage.

Prior to use, the proximal cap 4 and the needle cap assembly are removed.

FIG. 2 shows the housing sleeve 2. The housing sleeve has a housing window 11 through which the medicament container 3 is visible for the patient to inspect the content thereof.

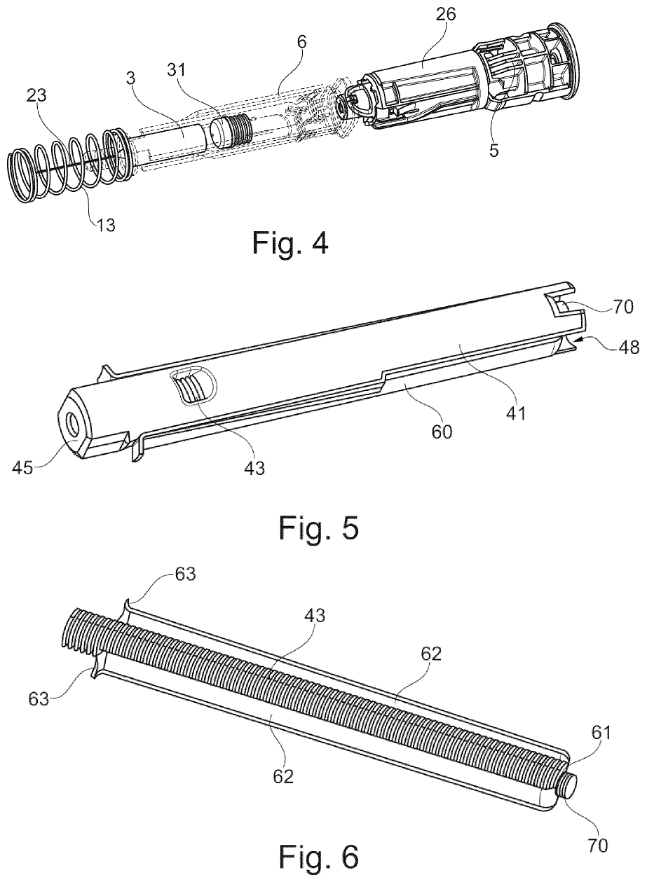

FIG. 3 depicts the so-called needle guard 10 (or ‘needle shield’ or ‘needle cover’). FIG. 4 shows the medicament delivery assembly of FIG. 1, with the proximal cap and the needle cap assembly removed, and without the housing sleeve 2 and the needle guard 10.

The assembly of FIG. 4 together with the needle guard 10 and the housing sleeve 2 thus constitutes the medicament delivery assembly immediately prior to its use.

In the depicted embodiment, the base unit of the medicament delivery device comprises, in addition to the housing sleeve 2 and the distal cap 5, also a container housing 6 accommodating the medicament container. The container housing is for example transparent or has a window, so as to allow the patient to inspect the medicament through the housing window 11. The container housing 3 in the depicted embodiment is a syringe that comprises a needle 23. Alternatively, the medicament container could be a container comprising a septum to be pierced, prior to use, by a needle that belongs to the medicament delivery device.

The medicament delivery device further comprises a drive locking member 26 (sometimes called ‘rotator’) and a plunger rod, together with further elements described in more detail hereinafter. The plunger rod, if applicable together with further parts moving with it, forms a drive unit.

As is known in the field, after removal of the proximal cap and the needle cap assembly, the needle guard 10 is forced, by a needle guard spring 13, into a proximal position, in which it protects the needle 23 of the medicament delivery assembly.

For activation, the patient presses the medicament delivery assembly of FIGS. 2-4, thus with the proximal cap and, if applicable, the needle cap assembly, removed, against the dose delivery site and thereby displaces the needle guard 10 towards distally relative to the housing, against a spring force of the needle guard spring 13. As a result, the needle 23 becomes exposed and can penetrate the patient's skin at the dose delivery site. At the same time, the needle guard 10 interacts with the drive locking member 26 to rotate the same about the proximodistal axis 20 (see FIG. 1), by a certain angle. The drive locking member 26 will, by this movement, release the drive unit and allow it to move axially into the proximal direction (expelling movement) so that the plunger rod acts on the medicament container's stopper 31 (or plunger). Thereby, it activates the medicament delivery.

There are several possible mechanisms by which a movement of an activation member—such as the needle guard 10—relative to the housing can activate the medicament delivery. Since the present invention does not depend on the nature of this activation, it is not described in any more detail here.

FIGS. 5-10 illustrate an embodiment of the first group of embodiments, being a first example how a delayed signal can be generated after medicament delivery.

FIG. 5 shows the plunger rod 41 together with the biasing member—here being a drive spring 43—, the signal member 60 and a delay member 70.

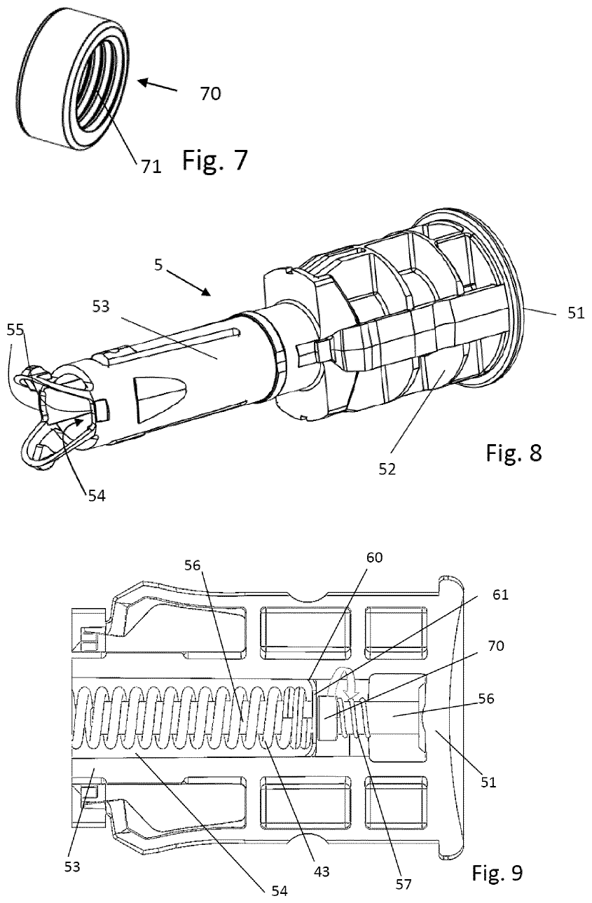

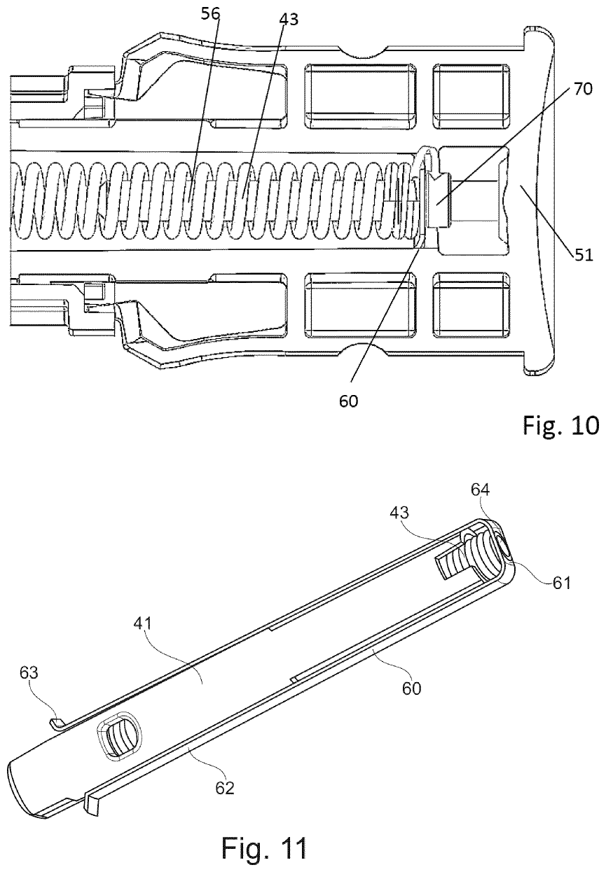

FIG. 6 depicts the elements of FIG. 5 without the plunger rod 41. FIG. 7 shows an enlarged view of the delay member 70. FIG. 8 shows the distal cap 5. FIGS. 9 and 10 depict the elements of FIG. 5 and the distal cap 5 as assembled together and in section, during two different stages at the end of the medicament delivery process.

The plunger rod 41 has the shape of a sleeve at least partially closed towards proximally by a proximal end bottom 45 that, during the expelling movement is pressed against the stopper 31 of the medicament container. The drive spring 43 extends into an interior of the plunger rod 41, with its proximal end abutting against the proximal end bottom 45.

The signal member 60 has the shape of a U-bracket, with a distal base 61 and two arms 62 extending towards proximally therefrom. At the proximal end of the arms 62, there is a radially outwardly extending, flange like support portion 63.

The distal cap 5 has a head portion 52 with a distal end wall 51. A tubular portion 53 having a cavity 54 open towards proximally extends into the head portion 52, so that the cavity is delimited, towards distally, by the distal end wall 51 (see FIG. 8 and FIG. 10). The distal cap further has a rod 56 extending from the distal end wall towards proximally. The rod 56 in the depicted embodiment extends through an orifice in the distal base 61 of the signal member 60 into the interior of the plunger rod 41, thereby also serving as spring guide for the drive spring 43.

The part of the rod 56 that is distally of the distal base 61 has a first section being a threaded section that comprises an outer thread 57, and second section distally thereof, the second, distal section having a smooth surface and not being threaded.

The delay member 70 has an inner thread 71. The outer thread 57 and the inner thread 71 together form a screw interface between the rod 56 and the delay member. The lead of the outer thread and of the inner thread is comparably high so that the screw interface is not self-locking but allows the delay member 70 to travel when subject to an axial force.

The drive spring 43 in the initial configuration, prior to the onset of the expelling movement is in an energy storage state, i.e. it is tensioned by being compressed between the distal base 61 of the signal member 60 and the proximal end bottom 45 of the plunger rod 41. The support portions 63 of the signal member 60 are proximally of the tubular portion 53 of the distal cap, whereby a proximal end face 55 of the tubular portion prevents the signal member 60 from being displaced, by the spring force of the drive spring 43, towards distally further into the cavity 54.

As soon as the drive locking member 26 (and/or other mechanism), due to an activation by the user, ceases to impede a movement of the plunger rod 41 towards proximally, the spring force of the drive spring 43 causes the expelling movement by driving the plunger rod 41 towards proximally, thereby acting on the stopper 31 of the syringe to expel the medicament via the needle 23. This movement towards proximally causes the plunger rod 41 to move out of the space between the arms 62 of the signal member 60.

The arms 62 of the signal member 60 are biased towards radially-inwardly, whereby they press against the outer surface of the plunger rod 41. As soon as the distal end portion 46 of the plunger rod 41 passes the distal end of the arms 62, the arms 62 are allowed to flex inwardly. Thereby, their support portions 63 get out of engagement with the proximal end face 55. The signal member 60 is thereby allowed to be moved towards distally by the spring force of the drive spring 43. The delay member 70 is initially in a proximal position in which it is in physical contact with, or close to, the distal base 61. Therefore, after the support portions 63 stop engaging the proximal end face 55, the drive spring 43 presses the signal member 60 against the delay member 70 and causes the same to rotate and to travel towards distally (see FIG. 9). This will cause the desired delay. Only after the outer thread 57 and the inner thread 71 get out of engagement (see FIG. 10) is the signal member 60 together with the delay member 70 allowed to accelerate, due to the spring force of the drive spring 43. The acceleration during the travel along the second section of the displacement path causes the delay member 70 to tap against the distal end wall 51 of the distal cap 5, to produce a click sound serving as the audible signal. Thus, the audible signal provides an audible or acoustic feedback. It should be noted that the audible signal may provide a tactile and/or haptic feedback as well to the user. The distal end wall hence acts as a stop structure for the signal member 60, via the delay member 70.

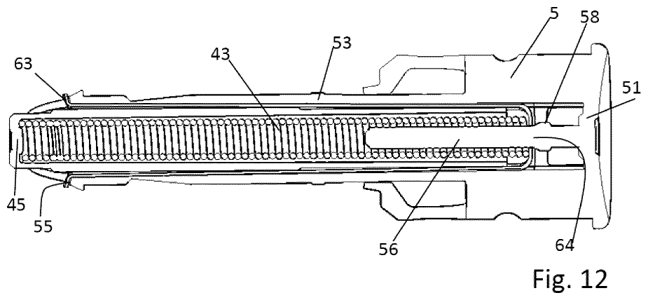

Elements of an embodiment of the second group, illustrating a second example of generating a delayed signal after medicament delivery, are shown in FIGS. 11 and 12. FIG. 11 displays a view of the plunger rod 41 with the drive spring 43 and the signal member 60, and FIG. 12 shows these elements assembled with the distal cap 5 in section. Only the differences to the first example are described.

In contrast to the first example, in the second example the medicament delivery device does not comprise a dedicated delay member. Rather, the rod 56 of the distal cap 5 has an enlarged area 58 that in the initial configuration, before the support portions 63 of the signal member 60 get out of engagement with the proximal end face 55 of the distal cap 5, is located immediately distally of the distal base 61. The orifice 64 in the distal base 61, through which the rod 56 extends, is slightly undersized with respect to the enlarged area 58.

The same way as in example 1, by the mechanism described hereinbefore, the signal member 60 is allowed to be moved towards distally by the spring force of the drive spring 43 as soon as the drive unit has reached the dispense position relative to the base unit. However, the enlarged area 58 interferes with this movement towards distally. Only due to a small elastic deformation of the signal member 60 around the orifice 64 (and/or potentially of the enlarged area 58) can the signal member's distal base 61 slide over the enlarged area, while the distal base 61 interacts with the enlarged area 58 to generate sufficient friction to substantially delay the movement but to not result in a complete interference fit.

After the distal base 61 has slid over the enlarged area 58, the signal member 60 may travel freely towards distally. During this movement of the distal base 61 along the second, distal section of the rod 56, the signal member 60 is accelerated until the distal base 61 taps against the distal end wall 51 of the distal cap 5, to produce a click sound serving as the—delayed—audible signal.

Various other embodiments may be envisaged.

For example, in embodiments of the first group of embodiments, the screw interface does not require both, an inner thread (for example of the delay member) and an outer thread (for example of the rod), as in the illustrated embodiment. Rather, it may be sufficient if one of the inner thread and of the outer thread is present, whereas the cooperating element just comprises at least one protrusion engaging, along the first section, with the inner/outer thread.

Further, not all of the elements described in the present text are necessarily separate parts. For example, the container housing and the housing sleeve and/or the housing sleeve and the distal cap could be integrated as one single part.

Instead of being integral with the distal cap 5, the rod also serving as the spring guide could be a separate member.

Moreover, the biasing member that stores the energy for the expelling movement is not necessarily a drive spring. It could for example be a gas cartridge or other simple energy store capable of setting the drive unit into motion to expel the medicament.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Various modifications to the embodiments described are possible and will occur to those skilled in the art without departing from the invention which is defined by the following claims.

1-13. (canceled)

14. A medicament delivery device for accommodating a medicament container, extending along a proximodistal axis, the medicament delivery device comprising:
a distal cap;
a plunger rod;
a signal member having a distal base and a plurality of arms extending proximally therefrom along the plunger rod;
a drive spring extending at least partially into an interior of the plunger rod, the drive spring having a proximal end abutting against a proximal end bottom of the plunger rod and a distal end abutting against the distal base of the signal member; and
a delay mechanism comprising:
a rod extending proximally in the cavity from the distal end wall, through an orifice of the distal base of the signal member; and
a delay member accommodated in the gap at least in the initial state, around a section of the rod,
wherein the signal member, the plunger rod and the biasing member are at least partially accommodated in a cavity of the distal cap, said cavity being open towards proximally, wherein in an initial state the drive spring is compressed and there is a gap between the distal base of the signal member and a distal end wall of the cavity, and wherein a release of the drive spring moves the plunger rod relative to the distal cap into a proximal direction, causing the plunger rod to act on the medicament container for expelling a medicament therefrom, and moves the signal member relative to the distal cap into a distal direction,

15. The medicament delivery device according to claim 14, wherein the rod comprises a first section having an outer thread, and the delay member comprises an inner thread interfacing the outer thread, wherein during the release of the drive spring, the distal movement of the signal member causes the delay member to rotate around the first section and move distally along the rod up to the distal end wall, causing an audible signal.

16. The medicament delivery device according to claim 15, wherein the rod comprises a second section that is non-threaded, between the distal end wall and the first section.

17. The medicament delivery device according to claim 14, wherein the delay member is a ring.

18. The medicament delivery device according to claim 14, wherein the delay member is an enlarged area of the section of the rod, wherein during the release of the drive spring, the distal movement of the signal member causes the orifice to slide over the delay member in a manner that friction is caused, the distal base moving distally along the rod up to the distal end wall, causing an audible signal.

19. The medicament delivery device according to claim 18, wherein the delay member is bulb-like and the orifice is circular.

20. The medicament delivery device according to claim 18, wherein the signal member comprises a plurality of cuts extending radially outwardly from the orifice.

21. The medicament delivery device according to claim 14, wherein in the initial state, the distal base of the signal member is in contact with the delay member.

22. The medicament delivery device according to claim 14, wherein the rod extends partially inside the drive spring.

23. The medicament delivery device according to claim 14, wherein the signal member has the shape of a U-bracket, and at a proximal end of each arm, the signal member has a radially outwardly extending, flange like, support portion.

24. The medicament delivery device according to claim 14, wherein the distal cap has a head portion comprising the distal end wall, and a tubular portion comprising the cavity.

25. The medicament delivery device according to claim 14, further comprising a drive locking member that, in the initial state, impedes a movement of the plunger rod towards proximally, and when activated, ceases to impede said movement, causing the drive springe to expand.

26. A medicament delivery assembly, comprising the medicament delivery device according to claim 14, and further comprising the medicament container assembled with the medicament delivery device.
